(19) United States
(12) Patent Application Publication
(10) Pub. No.: US 2012/0237539 A1
(43) Pub. Date: Sep. 20, 2012

(54) METHOD FOR MAKING TARGETED THERAPEUTIC AGENTS DIRECTED TO SOLUBLE TARGETS

(75) Inventors: Richard B. WILLIAMS, South Pasadena, CA (US); Robert GUERRERO, Pasadena, CA (US)

(73) Assignee: Proteonova, Inc., Pasadena, CA (US)

(21) Appl. No.: 13/455,598

(22) Filed: Apr. 25, 2012

Related U.S. Application Data
(63) Continuation of application No. PCT/US2010/054556, filed on Oct. 28, 2010.
(60) Provisional application No. 61/256,823, filed on Oct. 30, 2009.

(51) Int. Cl. C12P 21/00; A61P 37/04; A61K 39/00

(52) U.S. Cl. 424/192.1; 435/68.1

(57) ABSTRACT

Provided herein are methods for making targeted therapeutics that target soluble agents such as toxins, venoms, and factors that alter physiological biopathways as well as methods of using such therapeutics to treat patients or patient populations to reduce, eliminate, or inactivate, detrimental soluble agents that such patients or patient populations have been exposed to. In several embodiments the targeted therapeutics comprise a portion that interacts with a soluble agent of interest and a second portion that interacts with a subject's immune system, which enables treatment of a patient for exposure to the soluble agent.

### BACKGROUND

1. Field of the Invention

Several embodiments of the present invention relates to methods for making therapeutics tailored to soluble agents such as toxins, venoms, and factors that alter physiological biopathways as well as methods of using such therapeutics to treat patients or patient populations that have been exposed to such agents to reduce, eliminate, or inactivate, such detrimental soluble agents or their effects.

2. Description of the Related Art

Numerous toxins, venoms, chemical agents, and other agents that alter physiological biopathways exist and exposure to such agents presents a potentially significant health risk.

The immune system is involved in defending against these agents. Lymphocytes are critical to the immune system of vertebrates. Lymphocytes are produced in the thymus, spleen and bone marrow (adult) and represent about 30% of the total white blood cells present in the circulatory system of humans (adult). There are two major sub-populations of lymphocytes: T cells and B cells. T cells are responsible for cell-mediated immunity, while B cells are responsible for antibody production (humoral immunity). In a typical immune response, T cells are activated when the T cell receptor binds to fragments of an antigen that are bound to major histocompatibility complex (“MHC”) glycoproteins on the surface of an antigen presenting cell; such activation causes release of biological mediators (“interleukins”) which, in essence, stimulate B cells to differentiate and produce antibody (“immunoglobulins”) against the antigen. However, the immune system generally requires a first and a second exposure to an antigen (e.g., a soluble agent) in order to generate a response.

Some therapies are available for certain toxins. For example, antivenom against the venom particular snakes can be used to treat snake bites. The principle of antivenom is based on that of vaccines. Rather than inducing immunity in the patient directly, it is induced in a host animal and the hyperimmunized serum is transfused into the patient. Unfortunately, the number and variety of soluble toxins and agents is greater than the current host of therapies available to treat them.

Thus, there exists a need for methods of producing therapeutics that are specifically targeted against specific soluble targets and for methods of treating individuals or populations that have been exposed to such agents.

### SUMMARY

In various aspects, methods are provided herein for developing targeted therapeutics useful in treating a wide range of conditions by targeting soluble targets (e.g., toxins, venoms and the like). Methods are also provided herein that target soluble proteins without a pre-immunization of a subject with an epitope of the desired target. Also provided are methods for treating a disease or condition by administering a therapeutic produced by methods described herein.

In several embodiments, there is provided a method for generation of a bifunctional targeted therapeutic that targets a soluble agent, the method comprising identifying a first protein capable of interacting with a desired soluble target, identifying a first mRNA that encodes for the first protein, identifying an antigen capable of eliciting an immune response through interaction with one or more components of the immune system, identifying a second mRNA that encodes for the antigen, generating a first and a second cDNA corresponding to each of the first and the second mRNAs, fusing the first and the cDNA to generate a fused cDNA, translating the fused cDNA into a corresponding fused protein, wherein the a first portion of the fused protein is capable of interacting with the desired soluble target and a second portion of the fused protein is capable of eliciting an immune response, thereby generating a bifunctional targeted therapeutic that targets a soluble agent. In one embodiment, the antigen has a known mRNA sequence.

In several embodiments, there is provided a method for generation of a bifunctional targeted therapeutic that targets a soluble agent, the method comprising identifying a first protein capable of interacting with a desired soluble target, identifying a first mRNA that encodes for the first protein, identifying a second protein capable of eliciting an immune response through interaction with one or more components of the immune system, identifying a second mRNA that encodes for the second protein, generating a first and a second cDNA corresponding to each of the first and the second mRNAs, fusing the first and the cDNA to generate a fused cDNA, translating the fused cDNA into a corresponding fused protein, wherein the a first portion of the fused protein is capable of interacting with the desired soluble target and a second portion of the fused protein is capable of eliciting an immune response, thereby generating a bifunctional targeted therapeutic that targets a soluble agent. In some embodiments, the second protein is capable of binding to an antibody. In some embodiments, the second protein is capable of binding to the heavy chain of an antibody. In some embodiments, the second protein is capable of binding to the constant region of the heavy chain. In some embodiments, the second protein is capable of binding to the CH1 region of the heavy chain. In some embodiments, the antibody is an IgG antibody.

In several embodiments, the proteins are linked to their cognate mRNAs via a cross-linker. In one embodiment, the cross-linker is placed on a codon. In one embodiment, the cross-linker is placed on a pseudo-stop codon. In one embodiment, the cross-linker comprises a psoralen cross-linker, and wherein exposure of the mRNA to UV light links the mRNA to the protein. In some embodiments, the linker is selected from the group consisting of tRNA, modified tRNA, and tRNA analogs.

In some embodiments, the first protein is identified by screening a library comprising proteins linked to their cognate mRNAs to identify one or more proteins that interact with the soluble agent. In some embodiments, the method further comprises screening the bifunctional targeted therapeutic against the soluble agent and the one or more components of the immune system. In some embodiments, the fused cDNA comprises a bridge cDNA between the first and the second cDNA.

In some embodiments, the soluble agent is a selected from the group consisting of animal toxins, insect toxins, plant toxins, algae-derived toxins, fungi-derived toxins, bacterial-derived toxins, biowarfare agents, and biopathway modulators.

In some embodiments, the soluble agent targets one or more of the blood, blood vessels, nervous tissue, and muscle tissue.

In some embodiments, the soluble agent targets an ion channel.

In some embodiments, the soluble agent induces muscle paralysis.

In some embodiments, the soluble agent targets prevents blood clotting.

In some embodiments, the soluble agent induces increased gastrointestinal water secretion.

In several embodiments, there is provided a method for treating a subject that has been exposed to a soluble agent, comprising: identifying a subject who has been exposed to a soluble agent; and administering to the subject a bifunctional targeted therapeutic, wherein the immune response results in clearance of the soluble agent.

In several embodiments, there is provided a method for treating a subject that has been exposed to a soluble agent, comprising identifying a subject who has been exposed to a soluble agent, administering to the subject an antigen, wherein administration of the antigen induces production of antibodies directed to the antigen by the subject; administering to the subject the bifunctional targeted therapeutic comprising the antigen, wherein the administration allows the targeted therapeutic to bind to interact with the soluble agent and with the produced antibodies, wherein the interaction results in clearance the soluble target by the immune system, thereby treating the subject.

In several embodiments, there is provided a method for treating a subject that has been exposed to a soluble agent, comprising identifying a subject who has been exposed to a soluble agent, administering to the subject an antigen in order for the subject to produce antibodies to the antigen, identifying a first protein capable of interacting with the soluble agent, identifying a first mRNA that encodes for the first protein, identifying a second mRNA that encodes for the antigen, generating a first and a second cDNA corresponding to each of the first and the second mRNAs, fusing the first and the cDNA to generate a fused cDNA, translating the fused cDNA into a corresponding fused protein, administering the fused protein to the subject, wherein the administration allows the first portion of allows a first portion of the fused protein to interact with the soluble agent and a second portion of the fused protein to interact with the antibodies produced in response to the administration of the antigen, wherein the interactions result in the clearance of the soluble agent by the immune system, thereby treating the subject.

In several embodiments there is provided a method for treating a subject that has been exposed to a soluble agent, comprising, identifying a subject who has been exposed to a soluble agent, identifying a first protein capable of interacting with the soluble agent, identifying a first mRNA that encodes for the first protein, identifying a second protein capable of binding to an antibody, identifying a second mRNA that encodes for the second protein, generating a first and a second cDNA corresponding to each of the first and the second mRNAs, fusing the first and the cDNA to generate a fused cDNA, translating the fused cDNA into a corresponding fused protein, administering the fused protein to the subject, wherein the administration allows a first portion of the fused protein to interact with the soluble agent and a second portion of the fused protein to interact with the antibody. wherein the interaction with the antibody elicits an immune response, and wherein the immune response results in clearance of the soluble agent, thereby treating the subject.

In one embodiment, the antibody is an IgG antibody.

In several embodiments there is provided a use of bifunctional therapeutic generated according to the methods disclosed herein for the treatment of exposure to a soluble agent, wherein the exposure induces a deleterious effect in an exposed subject, and wherein the bifunctional therapeutic clears the soluble agent from the subject, thereby treating the exposure.

In several embodiments there is provided a use of a bifunctional therapeutic comprising a known antigen for the pre-treatment of a subject likely to be exposed to a soluble agent, wherein administration to the identified antigen induces the production of antibodies directed against the antigen, wherein subsequent actual exposure to the soluble agent induces the interaction of the first portion of the bifunctional protein with the soluble agent and the interaction of the produced antibodies with the second portion of the bifunctional protein, and wherein the interactions clear the soluble agent from the subject.

In several embodiments, methods provided herein utilize novel techniques for linking proteins to their corresponding mRNAs, and screening the protein-mRNA complexes for binding to a target associated with a particular soluble agent. In some embodiments, the identified proteins having high affinity for a soluble agent of interest are preferably isolated and linked to one or more immune modulators, to produce a variety of targeted therapeutics. Advantageously, the rapid and efficient identification, isolation, and production of proteins capable of recognizing targets of interest provides effective, low cost methods for the production of patient- and/or condition-specific therapeutics. In various embodiments, methods provided herein beneficially allow a wide range of soluble agents, and exposure thereto, to be treated with tailored therapeutics, within the context of existing health care budgets and resource allocations.

In various aspects, methods are provided herein for producing for treating exposure, or the possible exposure in the future to certain deleterious soluble agents, the therapeutics comprise a “targeting domain” that binds to all or a portion of a soluble agent, and a immune effector region capable of initiating an immune response. This “modular” architecture advantageously allows for the creation of uniquely targeted therapeutics by tailoring the targeting domain, which can then be used to enhance the efficacy of a variety of pre-existing or easily prepared therapeutic agents.

In various embodiments, the immune effector region of the therapeutic is linked, fused, or derivatized, directly or indirectly, to the soluble agent targeting domain to form the bifunctional therapeutic. In some embodiments, an immune effector is covalently linked to a target-binding domain, while in other embodiments it is non-covalently bound. In several embodiments, the target-binding domain and the immune effector binding domain can be directly linked, or indirectly linked, for example via a flexible linker peptide.

In other aspects, the invention provides methods for preparing a therapeutic for treating exposure to a soluble agent comprising isolating complexes of expressed mRNA molecules and their nascent polypeptides from an mRNA expression library; screening the protein-mRNA complexes for binding to all or a portion of a soluble agent; isolating and expressing mRNA encoding a protein that binds the target; and linking the target-binding protein (or derivatives, fragments or subunits thereof) to a therapeutic agent, such as an antibody capable of eliciting an immune response. In some embodiments, the preparation of the therapeutic further comprises allowing isolated mRNA encoding a target-binding domain to undergo in vitro evolution, selective mutagenesis, and/or other methods known in the art to identify and isolate mRNAs exhibiting stronger or more effective binding to the target, as described in more detail below.

In yet another aspect, the invention provides a kit for developing a bifunctional therapeutic for the treatment of (or pre-treatment of) exposure to a soluble agent.

In some embodiments, a kit is provided for developing patient-specific therapeutics for the treatment of soluble agent exposure, wherein the therapeutics are targeted to a unique marker, epitope, structural feature, etc., that is differentially expressed by the soluble agent (as compared to other molecules or compounds in the bloodstream.

These and other objects and features of several embodiments of the invention will become more fully apparent when the following detailed description is read in conjunction with the accompanying drawings.

### BRIEF DESCRIPTION OF THE DRAWINGS

FIGS. 1A-1D depict a scheme for development of therapeutics targeted to soluble targets. Panel A depicts identification of an mRNA linked to its cognate protein. A soluble target (e.g., a toxin or venom) on solid substrate is panned with a library of proteins (each of which may or may not interact with the soluble target) that are linked to their cognate mRNA. Panel B depicts identification of an mRNA associated with a known antigen, that antigen to be used to immunize a patient. Panel C depicts the fusion of the cDNAs from the mRNAs identified in each of Panels A and B. Panel D depicts the generation of a pool of bifunctional proteins directed to the soluble target, which are generated through translation of the cDNA with a linker provided between the two ends of the bi functional protein.

FIGS. 2A-2D depict a scheme for selecting bifunctional proteins that specifically react with the soluble target of interest and the known antigen used to immunize a subject. Panel A depicts the panning of the pool of bifunctional proteins against the soluble target which is affixed to a solid substrate. Panel B depicts specific binding of certain bifunctional proteins to the target of interest. Panel C depicts the reaction of the antigen portion of the bifunctional protein with a specific antibody bound to a substrate. Panel D represents the pool of generated bifunctional proteins that are to be used in treatments (e.g., those that have both a portion that reacts with the soluble target of interest and the known antigen).

FIGS. 3A-3C depict a schematic for the treatment of a subject who has been exposed to the soluble target. Panel 3A depicts the administration of the targeted therapeutic to the subject. Panel B represents the in vivo activity of the targeted therapeutic, e.g., the soluble target is bound to one end of the therapeutic and the other end of the therapeutic is bound by an antibody present in the subject. Panel C depicts the complex that is formed, which is then destroyed by the immune system.

FIGS. 4A-4D depict a schematic for development of therapeutics targeted to soluble targets to be administered to subjects who have not been pre-immunized with a known antigen. Panel A depicts identification of a first mRNA linked to its cognate protein. A soluble target (e.g., a toxin or venom) on solid substrate is panned with a library of proteins (each of which may interact with the soluble target) that are linked to their cognate mRNA. Panel B depicts an IgG molecule on a solid substrate that is panned with the library of proteins that are linked to their cognate mRNA, thereby identifying those proteins that react with the CH1 region of the antibody. Panel C depicts the generation of a fusion protein from cDNAs generated from each of the identified mRNAs. Panel D represents the pool of bifunctional proteins that are to be used in treatments (e.g., those that have both a portion that reacts with the soluble target of interest and the CH1 region of an antibody).

FIG. 5 depicts an example of the therapeutic agent generated by methods described herein.

FIGS. 6A-6D depicts a scheme for selecting bifunctional proteins that specifically react with the soluble target of interest and the CH1 region of an antibody. Panel A depicts the panning of the pool of bifunctional proteins against the soluble target which is affixed to a solid substrate. Panel B depicts specific binding of certain bifunctional proteins to the target of interest. Panel C depicts the reaction of the bifunctional protein with the CH1 region of antibodies bound to a substrate. Panel D represents the pool of generated bifunctional proteins that are to be used in treatments (e.g., those that have both a portion that reacts with the soluble target of interest and with the CH1 region of an antibody).

FIGS. 7A-7E depict the process in vivo that results in the clearance of the soluble target and generation of cells bearing antigen related to the soluble target. Panel A depicts a typical macrophage having an Fc receptor on its surface as well as the soluble target-bifunctional protein-antibody complex. Panel B depicts the binding of the complex to the macrophage. Panel C depicts the phagocytosis of the complex by the macrophage. Panel D depicts the digestion of the complex into small peptide fragments by enzymes. Panel E depicts the generation of a macrophage with the small peptides expressed on its surface, which will induce antibody production.

### DETAILED DESCRIPTION

Provided herein, are methods for producing targeted therapeutics that are tailored to specific soluble targets, as well as methods for treating subjects who have been exposed to such soluble targets. In several embodiments, the therapeutics are comprised of a “modular” architecture that allows a portion of the therapeutic to bind, engage, or otherwise interact the soluble target, and a second domain that binds, engages, or otherwise interacts with one or more components of a subject's immune system.

As used herein, the terms “bifunctional therapeutic”, “targeted therapeutic”, and “tailored therapeutic” shall be used interchangeably, shall be given their ordinary meaning and shall also refer to therapeutics with a soluble agent targeting portion and an immune effector portion (e.g., an antigen or an antibody binding region).

The terms “T lymphocyte” and “T cell” as used herein shall be given their ordinary meaning and shall also encompass any cell within the T lymphocyte lineage from T cell precursors to mature T cells.

The terms “B lymphocyte” and “B cell” shall be given their ordinary meaning and shall also encompass any cell within the B cell lineage from B cell precursors, such as pre-B cells, to mature B cells and plasma cells.

Immunoglobulin molecules consist of heavy (H) and light (L) chains, which comprise highly specific variable regions at their amino termini. The variable (V) regions of the H (V_{H}) and L (V_{L}) chains combine to form the unique antigen recognition or antigen combining site of the immunoglobulin (Ig) protein. The variable regions of an Ig molecule contain determinants (i.e., molecular shapes) that can be recognized as antigens or idiotypes.

The term “epitope” shall be given its ordinary meaning and shall also refer to the set of antigenic or epitopic determinants (i.e., idiotopes) of an immunoglobulin V domain (i.e., the antigen combining site formed by the association of the complementarity determining regions or V_{H} and V_{L} regions).

The term “idiotope” shall be given its ordinary meaning and shall also refer to a single idiotypic epitope located along a portion of the V region of an immunoglobulin molecule.

The term “immune effector” shall be given its ordinary meaning and shall also refer to a molecule, or derivatives, fragments, or subunits thereof, able to stimulate an immune response in the subject being treated, and may comprise an antibody, or derivatives, fragments, or subunits thereof, or a non-antibody molecule.

An “adjuvant” shall be given its ordinary meaning and shall also refer to a compound which enhances or stimulates the immune response when administered with one or more antigen(s).

“Malignant cells” shall be given its ordinary meaning and shall also refer to cells, which if left untreated, give rise to a cancer.

The terms “protein,” “peptide,” and “polypeptide” shall be given their ordinary meaning and shall also refer to a polymeric molecule of two or more units comprised of amino acids in any form (e.g., D- or L-amino acids, synthetic or modified amino acids capable of polymerizing via peptide bonds, etc.), and these terms may be used interchangeably herein.

The terms “soluble target” and “soluble agent” shall be given their ordinary meaning and shall also refer to toxins, venoms, factors that have the capacity to alter biochemical pathways, biochemical agents, and the like that are not solid or tissue-based (e.g., they present in the blood circulation of a subject as opposed to being a mass of cells, etc.). Non-limiting examples soluble targets are shown in Tables 1-8, below.

TABLE 1
Animal or Insect Toxins Targeting Blood and Blood Vessels

| General Target | Toxin Producers | Example Organism | Examples of Toxins Produced |
|---|---|---|---|
| Blood Vessel Dilators | Cephalopods | octopus | eledoisin |
| | | | tachykinin-like peptides |
| | Dipteran insects | mosquito | sialokinins |
| | Frogs | toad | tachykinin-like peptides |
| | Hymenopteran | bee | tachykinin-like peptides |
| | insects | | |
| | Short-tailed shrews | shrew | blarinatoxin |
| | | | kallikrein |
| | Toxicoferan reptiles | lizard | peptidase S1 toxins |
| Anticlotting Toxins | Dipteran insects | mosquito | aegyptin |
| | | | anopheline antiplatelet protein |
| | | | apyrase |
| | | | thrombostasins |
| | | | anophelins |
| | Assassin bugs | kissing bug | pallidipin and related lipocalins |
| | (reduviidae) | | |
| | | | infestin and related kazal-type |
| | | | proteins |
| | | | rhodnius prolixus aggregation |
| | | | inhibitor-1 |
| | Leeches | leech | leech antiplatelet protein |
| | | | saratin |
| | | | hirudins |
| | | | ornatins |
| | Snakes | rattlesnake | snake venom metalloproteinases |
| | | | C-type lectin toxins |
| | | | factor Xa-like proteases |
| | | | bothrojaracin |
| | Ticks | tick | moubatin and related lipocalins |
| | | | apyrase |
| | | | savignin and related kunitz-type |
| | | | proteins |
| | | | variegin |
| | | | variabilin |
| | Fleas | flea | apyrase |
| | Hymenopteran | bee | apyrase |
| | insects | | |
| | Caterpillars | wooly bear | lopap |
| | | | lonomin III |

TABLE 2
Animal or Insect Toxins Targeting Nervous and Muscle Tissue

| General | | Example | |
|---|---|---|---|
| Target | Toxin Producers | Organism | Examples of Toxins Produced |
| Calcium | Assassin bugs (reduviidae) | kissing bug | assassin bug toxins |
| Channel | Cone snails | cone snail | ω-conotoxins |
| Blockers | Lampreys | lamprey | lamprey salivary CRISP |
| | Snakes | rattlesnake | calcicludine |
| | Spiders | black widow | ω-neurotoxins |
| | Toxicoferan reptiles | lizard | CRISP toxins |
| Potassium- | Cone snails | cone snail | K-conotoxin |
| Channel | Hymenopteran insects | bee | apamin |
| Blockers | Scorpions | scorpion | short scorpion toxins |
| | Sea anemones | sea anemone | cnidaria kunitz-type proteinase |
| | | | inhibitors |
| | | | sea anemone type 3 potassium channel |
| | | | toxins |
| | Snakes | rattlesnake | dendrotoxins |
| | Spiders | black widow | K-atracotoxins |
| | Toxicoferan reptiles | lizard | CRISP toxins |
| Sodium- | Cone snails | cone snail | μ-O-conotoxins (pre- and post-synaptic) |
| Channel | Spiders | black widow | hainantoxins |
| Blockers | | | protoxin-II |
| | | | huwentoxin-IV |
| Sodium- | Scorpions | scorpion | β-toxins |
| Channel | Spiders | black widow | μ-neurotoxins |
| Activators | | | |
| Sodium- | Cone snails | cone snail | δ-conotoxins |
| Channel | Irukandji jellyfish | jellyfish | irukandji-toxins |
| Prolongers | Scorpions | scorpion | α-toxins |
| | Sea anemones | sea anemone | sea anemone sodium channel inhibitory |
| | | | toxins |
| | Spiders | black widow | δ-atracotoxins |
| Nicotinic | Cone snails | cone snail | α-conotoxins |
| Receptor | Snakes | rattlesnake | α-neurotoxins |
| Antagonists | | | |
| Muscarinic | Scorpions | scorpion | uncharacterized toxin or toxins |
| Receptor | Snakes | rattlesnake | three-finger toxins |
| Antagonists | | | phospholipase A2 toxins |

TABLE 3
Plant Toxins

| | | Symptoms Induced |
|---|---|---|
| Toxin Producers | Toxin Produced | by Toxin |
| Rosary pea | abin | inhibits protein |
| | | synthesis |
| Bitter almonds | amygdolin | β-glucosidosis in gut |
| (prunus dulcis) | | releases cyanide |
| Shikima plant | anisatin | respiratory paralysis |
| Nux-vomica tree | brucine | stryhnine-like toxin |
| Water hemlock | cicutoxin | CNS toxin |
| Delphinium | delphinine | cardiac activity toxin |
| Djenkon beans | djenkolic acid | kidney toxin |
| American mayapple | epipodophyllotoxin | cytotoxin |
| Gensing | falcarinol | contact dermatitis |
| Ivy | falcarinol | contact dermatitis |
| Gelonium plant | gelonin | protein synthesis toxin |
| Cotton plant | gossypol | dehydrogenase enzyme |
| | | inhibitor |
| Cassava leaves and roots | linamarin | gut releases cyanide |
| Lotus | lotaustralin | gut releases cyanide |
| Lima beans | lotaustralin | gut releases cyanide |
| Roseroot | lotaustralin | gut releases cyanide |
| White clover | lotaustralin | gut releases cyanide |
| Cycad seeds | β-methylamino-L- | neurotoxin |
| | alanine | |
| Hemlock water dropwart | oemanthotoxin | CNS toxin |
| Oleanders | oleandrin | neuro- and cardio- |
| | | toxins |
| Boraginaceae | pyrrolizidane alkaloids | liver toxins |
| Compositae | pyrrolizidane alkaloids | liver toxins |
| Orchidaceae | pyrrolizidane alkaloids | liver toxins |
| Leguminosiae | pyrrolizidane alkaloids | liver toxins |
| Euphorbia | resiniferatoxin | pain inducer |
| (resin spurge latex) | | |
| Castor bean | ricin | severe allergin |
| Foxglove | saponin (digoxine) | cardiac toxin |
| Nightshade datura | scopolamine | neurotoxin |
| Nux-vomica tree (seeds) | strychnine | neurotoxin |
| Locoweed | swainsonine | neurotoxin |
| Abyssinian kale | thionins | cytotoxin |
| Tutu plant | tututoxin | convolsant |

TABLE 4
Algae Derived Toxins

| General | | Toxin | |
|---|---|---|---|
| Target | Toxin Producers | Produced | Symptoms Induced by Toxin |
| Nervous | Dinoflagellates | saxitoxin | paralytic poisoning (in |
| Tissue | | | shellfish) |
| | Dinoflagellates | domoic acid | amnesic shellfish poisoning |
| | | | (in mussels, oysters, fish) |
| | Dinoflagellates | brevetoxin | neuro toxin (in shellfish) |
| | Dinoflagellates | okadaic acid | diarrhetic poisoning (in |
| | | | shellfish) |
| Hepatic | Blue green algae | microcystins | liver failure |
| Tissue | | | |

TABLE 5
Fungi Derived Toxins

| Toxin | Toxin | |
|---|---|---|
| Producers | Produced | Symptoms Induced by Toxin |
| Amanita | α-amanitin | deadly liver damage within 1-3 days |
| Amanitas | phallotoxin | gastrointestinal upset |
| Cortinarius | orellanine | deadly kidney failure within 3 weeks |
| Omphalotus | muscarine | respiratory failure, sometimes deadly |
| Gyromitra | gyromitrin | deadly neurotoxicity, GI upset, |
| | | destruction of blood cells |
| Coprinus | coprine | causes illness when consumed with |
| | | alcohol |
| A. muscaria | ibotenic acid, | hallucinogenic |
| | muscimol | |
| A. pantherina | ibotenic acid, | hallucinogenic |
| | muscimol | |
| A. gemmata | ibotenic acid, | hallucinogenic |
| | muscimol | |
| Psilocybe | psilocybin, | hallucinogenic |
| | psilocin | |
| Pleurotus | arabitol | gastrointestinal irritation |
| ostreatus | | |
| Boletus satanas | bolesatine | hemotoxin |
| Claviceps | ergotamine | affects vascular system, loss of limbs, |
| purpurea | | death |

TABLE 6
Bacterial Derived Toxins

| Toxin Producers | Toxin Produced | Symptoms Induced by Toxin |
|---|---|---|
| Bacillus anthracis | Anthrax toxin (EF) | edema and decreased phagocytic |
| | | response |
| Bordetella pertussis | Adenylate cyclase toxin (pertussis | formation of ion-permeable pores in cell |
| | AC) | membranes |
| Staphylococcus aureus | Alpha toxin | formation of ion channel in cell plasma |
| | | membrane |
| Vibrio cholerae | Cholera enterotoxin (Ctx) | secretion of water, electrolytes leading |
| | | to diarrhea |
| Escherichia coli | E. coli LT toxin | secretion of water, electrolytes leading |
| | | to diarrhea |
| Escherichia coli | E. coli ST toxins | secretion of water, electrolytes leading |
| | | to diarrhea |
| Shigella dysenteriae E. coli | Shiga toxin | diarrhea, hemorrhagic colitis, hemolytic |
| O157:H7 | | uremic syndrome |
| Clostridium perfringens | Perfringens enterotoxin | diarrhea |
| Clostridium difficile | ToxinA/ToxinB | cell necrosis, bloody diarrhea, colitis |
| Clostridium botulinum | Botulinum toxin | flaccid paralysis |
| Clostridium tetani | Tetanus toxin | spastic paralysis |
| Corynebacterium | Diphtheria toxin (Dtx) | inhibits protein synthesis in target cells |
| diphtheriae | | |
| Pseudomonas aeruginosa | Exotoxin A | inhibits protein synthesis |
| Bacillus anthracis | Anthrax toxin (LF) | cytotoxicity of cells |
| Bordetella pertussis | Pertussis toxin (Ptx) | interferes with metabolic regulation in |
| | | cells |
| Staphylococcus aureus | Exfoliatin toxin | intraepidermal separation |
| Staphylococcus aureus | Staphylococcus enterotoxins | causes massive activation of immune |
| | | system |
| Staphylococcus aureus | Toxic shock syndrome toxin (TSST- | inflammation, fever, shock (acts on |
| | 1) | vascular syst.) |
| Staphylococcus pyogenes | Erythrogenic toxin | inflammation, fever, shock |

TABLE 7
Biowarfare Agents

| Agent Target | Example Agent | Symptoms Induced by Agent |
|---|---|---|
| Nerve Agents | Cyclosarin (“GF”) | weakness, nausea; long-term neuro |
| | | functioning damage |
| | Sarin (“GB”) | weakness, nausea; long-term neuro |
| | | functioning damage |
| | Soman (“GD”) | vision, breathing difficulties; diarrhea; |
| | | fatal in large doses |
| | Tabun (“GA”) | vision, breathing difficulties; diarrhea; |
| | | fatal in large doses |
| | VX | vision, breathing difficulties; diarrhea; |
| | | fatal in large doses |
| | VR | breathing difficulties; slow heartbeat; |
| | | paralysis in large doses |
| | VM | vision, breathing difficulties; diarrhea; |
| | | fatal in large doses |
| | VG | vision, breathing difficulties; diarrhea; |
| | | fatal in large doses |
| | VE | vision, breathing difficulties; diarrhea; |
| | | fatal in large doses |
| | Insecticides (some) | |
| | Novichok agents | vision, breathing difficulties; diarrhea; |
| | | fatal in large doses |
| Blood Agents | Cyanogen chloride | respiratory problems; convulsions; fatal |
| | | in large doses |
| | Hydrogen cyanide | respiratory problems; convulsions; fatal |
| | | in large doses |
| | Arsines (most) | weakness, fatigue, nausea, paralysis or |
| | | death in large doses |
| Vesicant Agents | Sulfur mustard | skin & eye irritant, difficulty breathing; |
| | | abdominal pain |
| | Nitrogen mustard | skin & eye irritant, difficulty breathing; |
| | | abdominal pain; seizures |
| | Lewisite | eye, respiratory irritant; diarrhea; |
| | | seizures; low blood pressure |
| | Phosgene oxime | skin hives; severe lung and eye irritant |
| Pulmonary Agents | Chlorine | burning of eyes, nose, throat; nausea; |
| | | skin blisters; pulmonary edema |
| | Hydrogen chloride | corrosive burns in eyes, nose, throat, |
| | | upper respiratory tract |
| | Nitrogen oxides | eye, skin, respiratory irritation; |
| | | pulmonary edema |
| | Phosgene | burning of eyes, throat; shortness of |
| | | breath; nausea |
| Lachrymatory Agents | Tear gas | eye, nose, mouth, lung irritant; skin |
| | | burns; nausea |
| | Pepper spray | eye, nose, skin, respiratory irritant; |
| | | neurogenic inflammation |
| Incapacitating Agents | Agent 15 | confusion, hallucinations, blurred vision, |
| | | rapid heart rate |
| | Quinuclidinyl benzilate | confusion, hallucinations, blurred vision, |
| | | rapid heart rate |
| Cytotoxic Protein Agents | Ricin | respiratory distress, fever, nausea, |
| | | vomiting, diarrhea, organ failure |
| | Abrin | respiratory distress, fever, nausea, |
| | | vomiting, diarrhea, organ failure |

TABLE 8
Biopathway Modulators

| Factor | Biological Action |
|---|---|
| Interleukins | Tyrosine kinase inducers |
| Vascular Endothelial Growth Factors | Cancer and other disorders |
| (VEGF, A, B, C, D, E, F) | |
| Placental Growth Factor | Cancer angiogenesis/ |
| (PLGF) | vasculogenesis |
| Tumor Necrosis Factor Alpha | Inflammation, arthritis |
| Tumor Necrosis Factor Beta | Kills infected cells, product of |
| | CD8⁺ T cells |
| CC Chemokine | Cell migration inducer |
| (β-Chemokine) | |
| CC Chemokine ligands | Migration inducer for monocytes, |
| (CCL-1, 15, 23, 28) | NK and dendritic cells |
| Monocyte Chemo attractant | Inducer of monocytes to become |
| Protein 1 (MCP- 1 or CCL2) | tissue macrophages |
| RANTES (CCL-5) | Attract CCR6 expressing T cells, |
| | eosinophils and basophils |
| Glutamic Acid-leucine-arginine | Inducer of migration of neutrophils |
| (ELR) | |
| CXCL13 | Chemo attractant for lymphocytes |
| Lymphotactin Alpha | Attract T cell precursors to thymus |
| (XCL-1) | |
| Lymphotactin Beta | Attract T cell precursors to thymus |
| (XCL-2) | |
| Fractalkine | Adhesion molecule |
| (CX₃CL1) | |
| Interferon Type I, II, & III | Anti viral and cancer, activate |
| | Nk cells and macrophages |

It shall be appreciated, based on the non-limiting examples of soluble agents presented herein that the variety of soluble agents and the potential deleterious effects that they induce is quite broad. However, in several embodiments the methods and therapies disclosed herein allow the rapid, cost-effective, and specific generation and use of bifunctional therapeutics that reduce, eliminate or otherwise diminish the deleterious effects of exposure to such soluble agents.

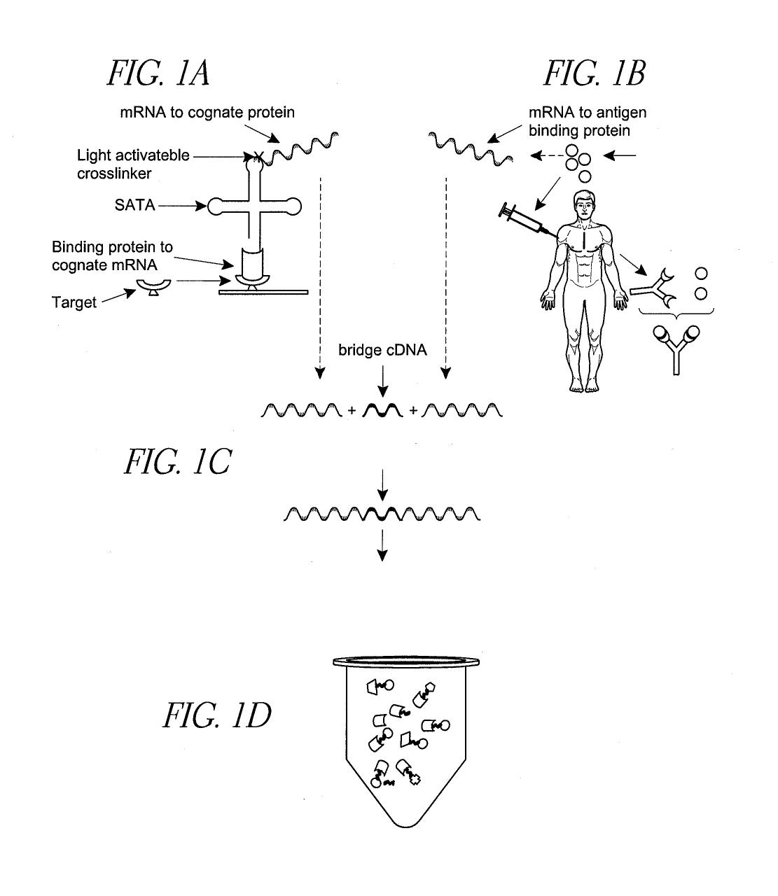

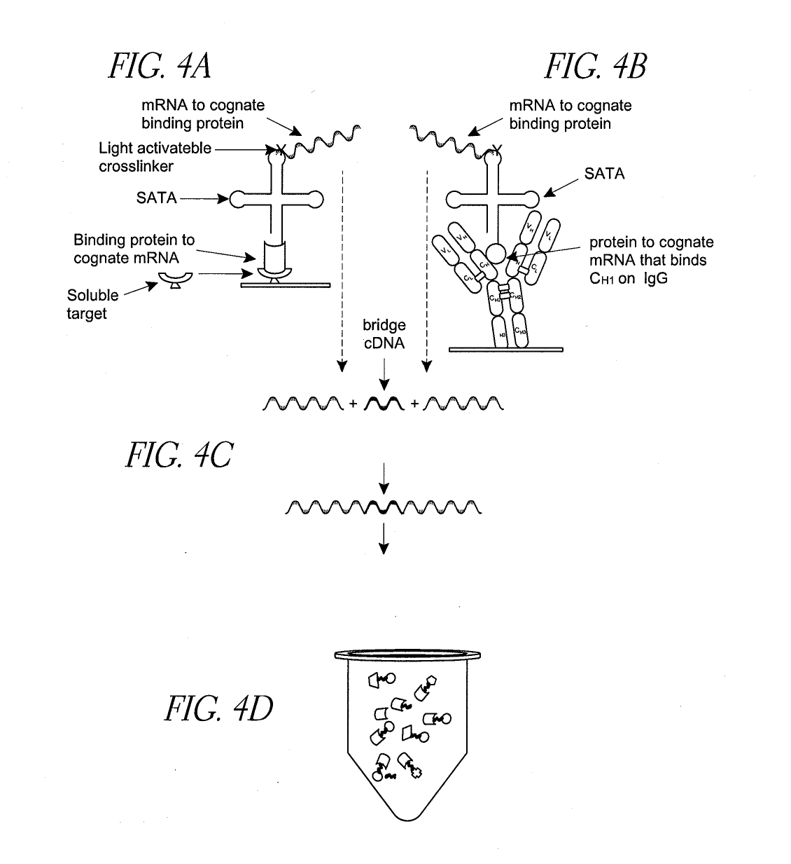

In several embodiments, the generation of a bifunctional targeted therapeutic is performed based on the exploitation of a known antigen and a sequence of mRNA that encodes that antigen (see e.g., FIGS. 1A-1D). In several embodiments, a more general immune-mediated approach is used to generate the therapeutics (see e.g., FIGS. 4A-4D). However, in both such approaches, a specific protein that interacts with the soluble agent is identified. Thus, the bifunctional (one portion interacts with the soluble target while another interacts with one or more components of the immune system) protein functions to snare the soluble target and simultaneously (or subsequently) clear the soluble agent from a subject via an immune-mediated response.

In several embodiments, the bifunctional targeted therapeutics are particularly advantageous because no therapy or treatment means for clearing the agent presently exists. In some embodiments, the selectivity the bifunctional targeted therapeutics enhances the efficacy of treatment relative to non-tailored therapeutics, due, for example, to the non-selective activity of non-tailored therapeutics. In some embodiments, the therapeutics provided are more efficient at interacting with a wider variety of soluble agents as the protein-target interaction presents a wider scope of interactions that can be exploited. For example, use of an antibody based approach against certain soluble agents may not be particularly effective against agents having a low immunogenicity. In contrast, the use of targeted therapeutics provide for herein can, in some embodiments, exploit protein-protein interactions between the soluble agent and the protein therapeutic (e.g., a steric relationship that is not highly immunogenic). As such, the possible ways of capturing or interacting with the soluble agent may be greater, and in some embodiments, more effective, than simply relying on antibody-based interactions. In additional embodiments, proteins that interact with soluble chemical agents are used in the generation of the bifunctional protein.

### Generation of Bifunctional Targeted Therapeutics

### Therapeutics Exploiting a Known Antigen

In various embodiments, the generation of bifunctional therapeutics provided herein for binding to a variety of soluble agents is made possible by utilizing novel methods for linking proteins to their corresponding mRNAs (as “cognate pairs”). In some preferred embodiments, protein libraries are prepared comprising a large number of cognate pairs, and the libraries are screened for cognate pairs that bind to a target of interest, such as the individualized targets described herein. Further information regarding the generation and use of a library of proteins linked to their cognate mRNAs can be found in U.S. Pat. Nos. 6,962,781; 7,351,812; 7,410,761; and 7,488,600 and the following U.S. patent application Ser. No. 11/813,849, filed May 2, 2008 (currently pending) and Ser. No. 12/525,437, filed Jul. 31, 2009 (currently pending). Each of the aforementioned Patents and patent applications are expressly incorporated in their entirety by reference herein. As shown generally in FIGS. 1A-1D, several embodiments, bifunctional targeted therapeutics are produced that will be used in conjunction with a known antigen. In FIG. 1A, a soluble target is shown on a solid substrate that is panned with protein-SATA-mRNA library under neutral and then acid conditions, stimulating —plasma and endosome conditions. As shown in FIG. 1A, a soluble target of interest is immobilized on a solid substrate and panned with a library of proteins that are linked to their cognate mRNA (e.g., the mRNA sequences that code for that particular protein). Entire proteins need not be used, fragments, or portions of proteins are sufficient to interact with a soluble target in some embodiments. In several embodiments, the library is panned against the soluble target under conditions similar to those in the bloodstream (e.g., about a neutral pH). In several embodiments, the library is also panned against the soluble target under acidic conditions, simulating the endosome or other cellular complex in which the degradation of the soluble target may occur. Based on the panning of the library a particular protein (or protein fragment) that demonstrates an interaction with the soluble target may be selected.

In FIG. 1B, an antigen with known code for its mRNA is used to immunize patient(s). The immunized patient makes antibodies to the antigen. As shown in FIG. 1B, an antigen that is known is selected. In some embodiments, the antigen is one to which a subject has already been exposed. In some embodiments, the known antigen is administered to the subject, in order to “vaccinate” the subject and induce the generation of antibodies against that antigen. The advantage of using a known antigen are that an mRNA sequence known to encode that antigen may already be known, thereby reducing the need to pan an additional protein-mRNA library to generate the portion of the therapeutic that will elicit an immune response. However, in some embodiments, a second panning step is used.

In FIG. 1C, the two mRNAs, converted to cDNA and PCR amplified, are then enzymatically fused with a bridge cDNA between the two cDNAs. As shown in FIG. 1C, the mRNA encoding the protein that interacts with the soluble target and the mRNA that encodes the known antigen are, in some embodiments, converted to cDNA by a reverse transcription reaction. In some embodiments, a linker or bridge sequence of cDNA is ligated between the two cDNAs, and then the entire sequence is amplified by polymerase chain reaction. In some embodiments, the bridge cDNA encodes a sequence of known hydrophilic amino acids. In some embodiments, the bridge (or other portion of the cDNAs) encode a series of amino acids that can be later used to purify the resultant protein (e.g., a histidine tag, allowing for well-established nickel-sepharose chromatography methods). In some embodiments, the individual cDNAs are first amplified and then ligated with the bridge cDNA. Regardless, of the order of amplification and ligation, the resultant sequence of cDNA (now comprising a cDNA sequence for the protein that interacts with the soluble target and a cDNA sequence that encodes the known antigen and a bridge cDNA sequence) is then, in some embodiments, translated into protein.

In still additional embodiments, the individual cDNAs are translated into protein and later linked together to form the bifunctional therapeutic. In some embodiments, a protein coupling agent, such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexaned-iamine), bis-diazonium derivatives (such as bis-(p-diazoniumbenzoyl)-ethyl-enediamine), diisocyanates (such as tolyene 2,6-diisocyanate), or bis-active fluorine compounds (such as 1,5-difluoro-2,4-dinitrobenzene), is used to link two or more protein components comprising the therapeutic. In some embodiments, such an approach is particularly advantageous as a variety of pools of soluble agent binding domains and antigenic domains can be prepared, stored, and later used in various combinations depending on the needs of a particular subject. In some embodiments, the translation is in vitro, while in some embodiments, in vivo translation is used, followed by isolation of the resultant protein (e.g., by gel electrophoresis, size exclusion, selective tag purification, or any other means known in the art to purify proteins).

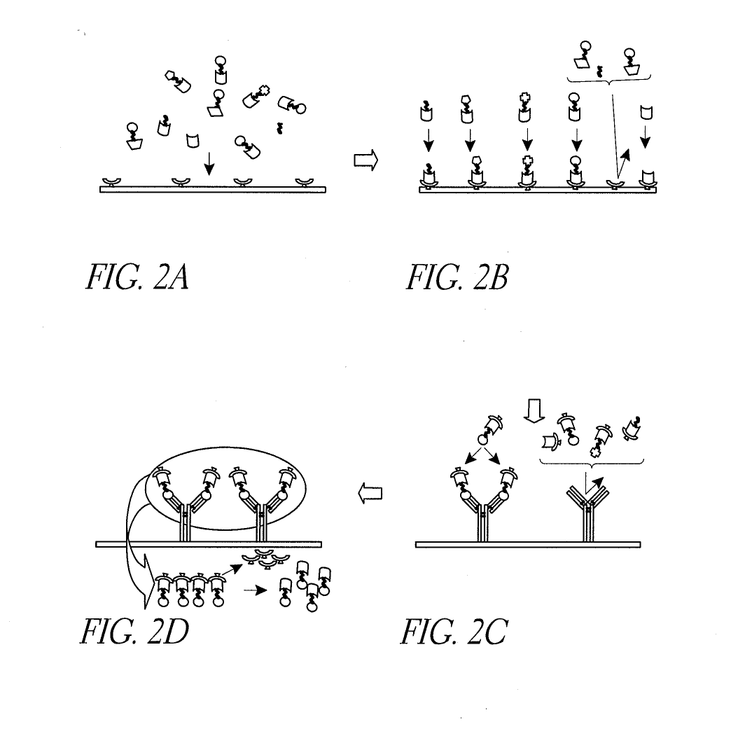

After generation of a putative bifunctional therapeutic, a series of panning steps are performed, in some embodiments, to identify and select for therapeutics that are more efficient at interacting with the soluble agent and/or an antibody. As shown in FIG. 2A, a pool of bifunctional therapeutic candidate molecules are panned (e.g., reacted with) the soluble target of interest, which is bound to a solid substrate. The candidate molecules are represented by the small geometric, semi-rectangular geometric shapes (the portion which may interact with a soluble target) a linker portion (the curvy line) and a rounded portion (represents the antigen domain, which will interact with an antibody generated by the “vaccinated” subject). As shown in Panel B, the bifunctional proteins that recognize the soluble target will bind with it; those that do not will be removed by washing (or other similar removal step). Those that bind with the soluble target are collected and the soluble target is removed (e.g., by incubation or washing in detergent-rich buffers), in some embodiments, re-reacted with unbound soluble target. In several embodiments, such an approach assures that the soluble target does not cause steric hindrance changes in the bifunctional protein that affect the reaction with the antibody. As shown in Panel C, the target bound bifunctional protein is reacted with antibodies that are directed to the known antigen portion of the bifunctional protein. In several embodiments, the antibodies that bind the antigen portion of the bifunctional proteins are affixed to a solid substrate. Only bifunctional protein candidates with the correct immunizing antigen will bind with the idiotype site of the antibodies while those with other antigens will not, and can be removed (e.g., washed) from the reaction site). As represented in Panel D, only the bifunctional proteins with both correct ends are collected. After removal of the soluble target component, these bifunctional proteins become the therapeutic. In other embodiments, other methods are used to determine if the candidate therapeutics interact with the soluble target and/or the generated antibody, for example Western blot, immunoprecipitation, ELISA, and FACS analyses using, as appropriate, Fab idiotype fragments, peptides, idiotype-expressing cells or extracts thereof.

Therapeutics with Direct Antibody Binding Regions

As shown in FIGS. 4A-4D, in some embodiments, a bifunctional therapeutic that comprises a region that interacts with the soluble target and a region that binds directly to the structural portions of an antibody are generated. As shown in FIG. 4A, the soluble target on solid substrate is panned with a protein-SATA-mRNA library under both neutral and acid conditions to stimulate plasma and endosome conditions, similar to that described above. In FIG. 4B, IgG on solid substrate is panned with protein-SATA-mRNA library under both neutral and acid conditions to simulate plasma and endosome conditions. For both panning methods, both neutral and acidic conditions are used to simulate both plasma and endosome-like environments. In FIG. 4C, the two mRNA's are converted to cDNA and PCR amplified. then enzymatically fused with a bridge cDNA between them. The fused, cDNA is then PCR amplified and transcribed to mRNA and translated in vitro, and screened for intact bifunctional proteins.

In some embodiments, the antibody panned is an IgG, though IgM, IgA, or IgE isotypes are used in some embodiments. In some embodiments, using IgG isotypes, isotype 1, 2, 3, or 4 can be used, depending on the embodiment. In some embodiments, the optimum binding protein-IgG isotype combinations will be made empirically, while in others, the isotype can be selected ahead of time. In some embodiments, proteins that bind to the variable regions are identified by panning and used in generation of the bifunctional therapeutic. In several embodiments, those proteins that bind to the constant region of an antibody are used. In some embodiments, the constant heavy chain is the preferred binding site of proteins to be used in generating the therapeutic.

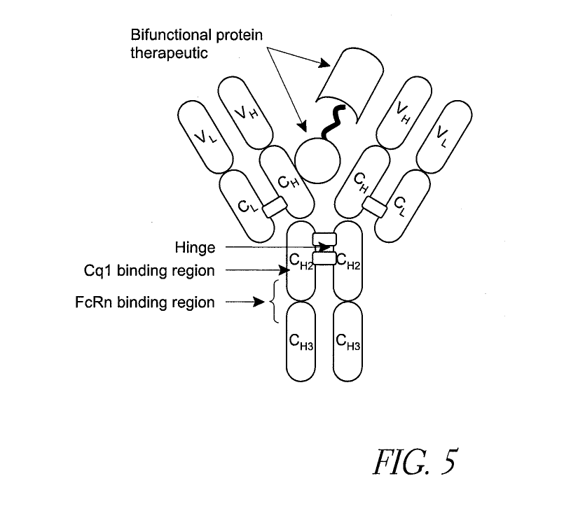

FIG. 5 depicts a representative targeted therapeutic that is consistent with several embodiments disclosed herein.

Several embodiments utilizing IgG are particularly advantageous due to the presence of the FcRn binding site on the IgG. The FeRn binding site is located between the C_{H2} and C_{H3} regions of the Fc stem of the IgG, provides a salvage pathway for returning IgG to the circulation. As such, use of IgG antibodies provide a longer residence time in the circulation, and, in some embodiments, an increased therapeutic time.

As discussed above, the selections, in some embodiments, are performed at both neutral and acid pH (pH 5-6.2) to ensure that the bifunctional protein will remain bound to the soluble target while in the endosome.

The remainder of the generation of the targeted therapeutic is performed as described above, with the exception, of course, that one portion of the therapeutic will bind directly to a portion of an antibody that preexists in a subject. As such, this approach advantageously obviates the need for vaccination of the subject with a known antigen prior to administration of the targeted therapeutic.

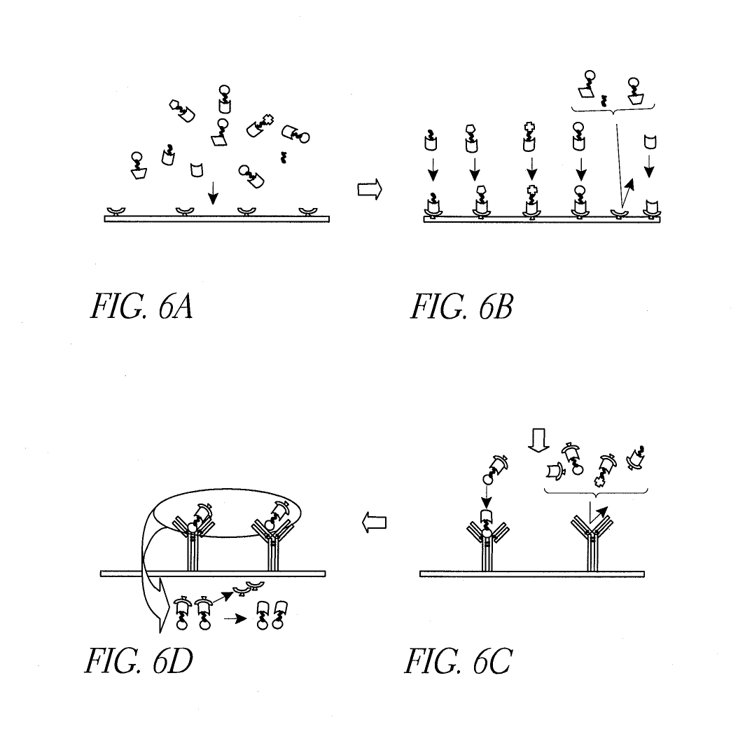

In FIG. 6A, bifunctional protein candidates are panned against soluble target affixed on a solid substrate. In FIG. 6B, the bifunctional proteins that recognize the soluble target will bind with it, and those that do not recognize the soluble target will not bind. Those that bind are collected, and then re-reacted with soluble target. This assures that the soluble target does not cause steric hindrance changes in the bifunctional protein that might affect the reaction with the CH1 portion of the antibody. In FIG. 6C, the bifunctional protein candidates pre-reacted with soluble target are panned against IgG affixed to solid substrate. Bifunctional protein candidates that recognize the CH1 portion of the IgG specifically will bind, and those that do not recognize the CH1 portion will not bind. In FIG. 6D, only the bifunctional proteins with both correct ends are collected. With removal of the soluble target component, they can become therapeutic.

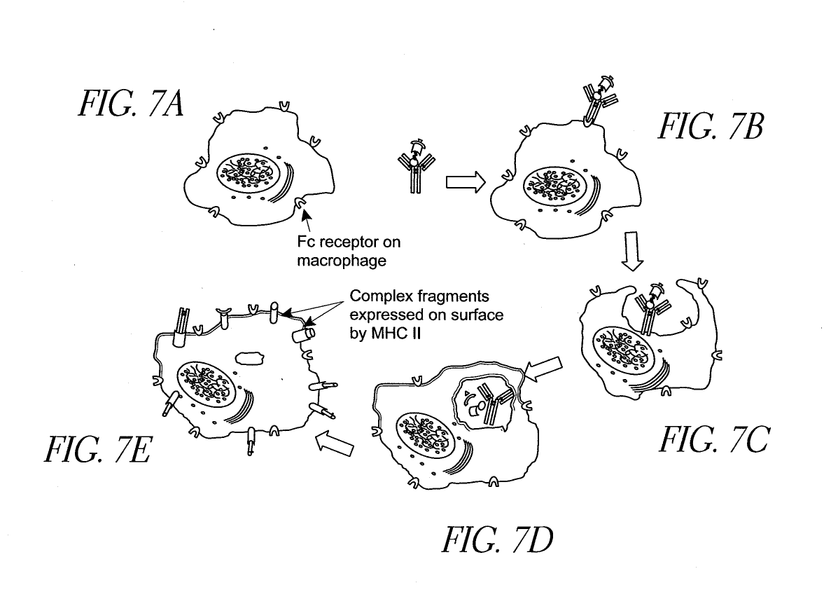

Screening of candidate bifunctional proteins is likewise performed as described above, with the exception that the antibody that is panned is matched to the antibody isotype that the mRNA used to generated the bifunctional protein encoded (e.g., IgG with IgG). See, for example FIGS. 7A-7E.

### Administration of Bifunctional Targeted Therapeutics

### Therapeutics Exploiting a Known Antigen

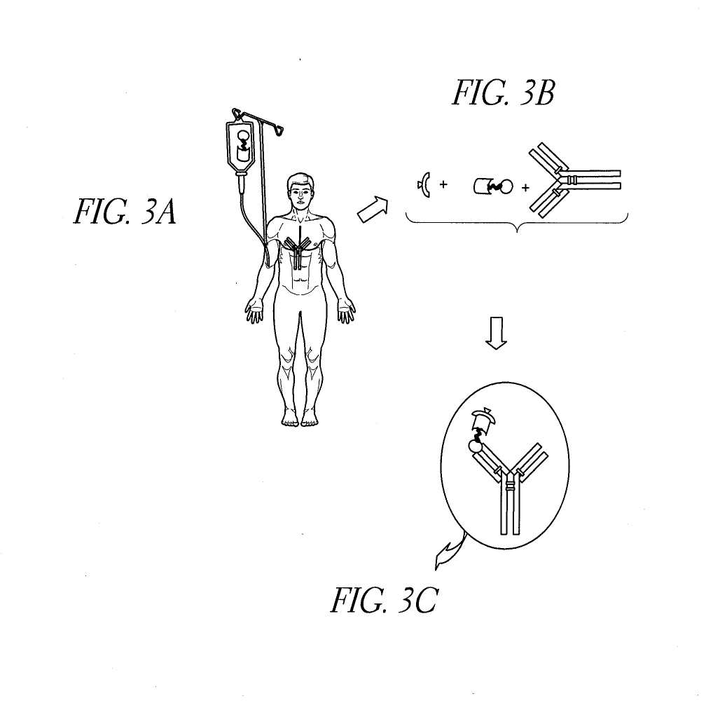

In several embodiments, the initiation of therapy with bifunctional therapeutics that exploit known antigens occurs when a subject who has been exposed to a soluble target is administered the known antigen. As discussed above, an mRNA that encodes the known antigen will be used in the generation of the bifunctional therapeutics. The pre-administration of the known antigen to the subject acts to induce generation of antibodies directed against that antigen. After the screening of the candidate bifunctional therapeutics and identification of one (or more) that appropriately react with both the soluble target and the antibody, the identified bifunctional therapeutics are administered to the subject. FIG. 3A shows the therapeutic bifunctional protein being infused into the patient. As shown in FIG. 3B, the “target” side of the bifunctional therapeutic binds with the soluble target. The antigen portion of the bifunctional therapeutic is bound by the patient's own antibodies against the antigen. As seen in FIG. 3C, the result of the two reactions is that the soluble target which is bound indirectly via the bifunctional therapeutic to the antibodies is flagged for destruction by the immune system, thereby removing the target from the individual.

Therapeutics with Direct Antibody Binding Regions

In contrast to the use of the bifunctional therapeutic proteins wherein one portion of the protein comprises an antigen against which the patient has been immunized, the administration of therapeutics with a region that directly binds to a portion of an antibody dose not need to be preceded by vaccination of the subject with a known antigen. Thus, in several embodiments, a subject who has been exposed to a soluble target is identified. As discussed above, a bifunctional therapeutic is generated that is directed specifically to the soluble target and also to a specific portion of an antibody (e.g., an IgG). After screening the candidate pool, the selected bifunctional therapeutics are administered to a subject. In the subject's circulation, the target portion of the bifunctional protein interacts with the soluble target. Similarly, the antibody binding portion interacts with a circulating antibody. In several embodiments, the antibody binding portion binds to the heavy chain of an IgG within the constant region (e.g., the C_{H1} binding site). The schematic for the subsequent reaction in vivo is illustrated in FIG. 7. As shown in FIG. 7A, macrophages express Fc receptors on their surface. In several embodiments, after administration of the bifunctional therapeutic, the Fc domain of the IgG complexes bound to the bifunctional therapeutic will bind the Fc receptors on the macrophages (FIG. 7B). The bifunctional complex is subsequently phagocytosed (FIG. 7C). After phagocytosis, the bifunctional therapeutic will be digested to small peptide fragments by enzymes in the endosome, lysosome or other acid vesicle to which the bifunctional therapeutic is trafficked. While in the lysosome (or other vesicle), in several embodiments, the resultant fragments are associated with MHC II and are subsequently presented on the surface of the macrophage. Once on the surface, they are recognized by CD4⁺ T cells that express the appropriate T cell receptor. Binding of the fragment, which is now functionally an antigen, and CD4 to the MHC II activates the T cells to secrete cytokines (interleukins) that activate B cells. The activated B cells undergo proliferation and produce antibodies against the antigen fragment. Thus, in several embodiments, the single administration of the bifunctional therapeutic induces a last therapeutic immune response. However, in some embodiments, both for the direct antibody binding therapeutic and the antigen encoding (pre-immunization) therapeutic, two, three, four, five, or more administrations may be given in certain embodiments.

While a number of preferred embodiments of the current invention and variations thereof have been described in detail, other modifications and methods of use will be readily apparent to those of skill in the art. For all of the embodiments described above, the steps of the methods need not be performed sequentially. Accordingly, it should be understood that various applications, modifications and substitutions may be made without departing from the spirit of the invention or the scope of the claims.

What is claimed is:

1. A method for generation of a bifunctional targeted therapeutic that targets a soluble agent, the method comprising:
identifying a first protein capable of interacting with a desired soluble target;
identifying a first mRNA that encodes for said first protein;
identifying an antigen capable of eliciting an immune response through interaction with one or more components of the immune system;
identifying a second mRNA that encodes for said antigen;
generating a first and a second cDNA corresponding to each of said first and said second mRNAs;
fusing said first and said cDNA to generate a fused cDNA;
translating said fused cDNA into a corresponding fused protein,
wherein said a first portion of said fused protein is capable of interacting with said desired soluble target and a second portion of said fused protein is capable of eliciting an immune response,
thereby generating a bifunctional targeted therapeutic that targets a soluble agent.

2. The method for generation of a bifunctional targeted therapeutic according to claim 1, wherein said antigen is a second protein.

3. The method according to claim 1, wherein said first protein is identified by screening a library comprising proteins linked to their cognate mRNAs to identify one or more proteins that interact with the soluble agent.

4. The method according to claim 1, further comprising screening the bifunctional targeted therapeutic against said soluble agent and said one or more components of the immune system.

5. The method according to claim 1, wherein said fused cDNA comprises a bridge cDNA between said first and said second cDNA.

6. The method according to claim 1, wherein said antigen has a known mRNA sequence.

7. The method according to claim 2, wherein said second protein is capable of binding to the heavy chain of an antibody.

8. The method according to claim 7, wherein said second protein is capable of binding to the constant region of the heavy chain.

9. The method according to claim 8, wherein said second protein is capable of binding to the CH1 region of the heavy chain.

10. The method according to claim 1, wherein said second protein is capable of binding to an IgG antibody.

11. The method according to claim 1, wherein said soluble agent is a selected from the group consisting of animal toxins, insect toxins, plant toxins, algae-derived toxins, fungi-derived toxins, bacterial-derived toxins, biowarfare agents, and biopathway modulators.

12. The method according to claim 1, wherein said soluble agent targets one or more of the blood, blood vessels, nervous tissue, and muscle tissue,
wherein said soluble agent targets one or more ion channels,
wherein said soluble agent induces muscle paralysis,
wherein said soluble agent prevents blood clotting, or
wherein said soluble agent induces increased gastrointestinal water secretion.

13. The method according to claim 3, wherein said protein is linked to its cognate mRNA via a cross-linker.

14. The method of claim 13, wherein the cross-linker is placed on a codon.

15. The method of claim 14, wherein the cross-linker is placed on a pseudo-stop codon.

16. The method of claim 13, wherein the cross-linker comprises a psoralen cross-linker, and wherein exposure of the mRNA to UV light links said mRNA to said protein.

17. The method of claim 13, wherein said linker is selected from the group consisting of tRNA, modified tRNA, and tRNA analogs.

18. A method for treating a subject that has been exposed to a soluble agent, comprising:
identifying a subject who has been exposed to a soluble agent; and
administering to said subject the bifunctional targeted therapeutic generated by the method of claim 1,
wherein said immune response results in clearance of the soluble agent.

19. A method for treating a subject that has been exposed to a soluble agent according to claim 18, further comprising:
administering to said subject an antigen prior to administering said bifunctional targeted therapeutic, wherein administration of said antigen induces production of antibodies directed to said antigen by said subject;
wherein said administration allows the targeted therapeutic to bind to interact with said soluble agent and with said produced antibodies.

20. The method according to claim 18, wherein said antibody is an IgG antibody.
